(19) United States
(12) Patent Application Publication
(10) Pub. No.: US 2026/0220768 A1
(43) Pub. Date: Jul. 30, 2026

(54) MEDICAL AI FOUNDATION MODEL FOR MULTI-FINDING ABNORMALITY DETECTION

(71) Applicant: Siemens Healthineers AG, Forchheim (DE)

(72) Inventors: Youngjin Yoo, Princeton, NJ (US); Eli Gibson, Lawrenceville, NJ (US); Gengyan Zhao, Robbinsville, NJ (US); Bogdan Georgescu, Princeton, NJ (US)

(21) Appl. No.: 19/036,495

(22) Filed: Jan. 24, 2025

(51) Int. Cl. G06T 7/00; G06F 40/289; G06V 10/762; G06V 10/774; G06V 10/82; G16H 50/20

(52) U.S. Cl. CPC G06T 7/0012; G06F 40/289; G06V 10/762; G06V 10/774; G06V 10/82; G16H 50/20; G06T 2207/20081; G06T 2207/20084; G06T 2207/30004; G06V 2201/03

(57) ABSTRACT

Systems and methods for training a medical foundation model are provided. Image/text pairs each comprising a training medical image and training text-based medical data are received. The training medical images are encoded into image features using a machine learning based image encoder. The training text-based medical data are encoded into text features using a machine learning based text encoder. Clinical labels are extracted from the training text-based medical data using one or more language models. At least one of the machine learning based image encoder or the machine learning based text encoder are trained based on the image features, the text features, and the clinical labels.

### TECHNICAL FIELD

The present invention relates generally to AI/ML (artificial intelligence/machine learning) based medical imaging analysis, and in particular to a medical AI foundation model for multi-finding abnormality detection.

### BACKGROUND

AI/ML (artificial intelligence/machine learning) based models have been used for performing various medical imaging analysis tasks, such as, e.g., automated diagnosis, classification, and segmentation. Such AI/ML based models are typically trained for performing a specific medical imaging analysis task based on large scale training datasets using supervised or self-supervised learning methods. Recently, large-scale, pre-trained medical foundation models have been proposed for performing a wide array of medical imaging analysis tasks. Medical foundation models are typically used for extracting features from medical images and the extracted features are used for performing one or more medical imaging analysis tasks. However, training medical foundation models for extracting features that accurately represent various pathologies and clinical findings in medical images is a complex task.

### BRIEF SUMMARY OF THE INVENTION

In accordance with one or more embodiments, systems and methods for performing one or more medical imaging analysis task using a medical foundation model are provided. Medical data of a patient is received. Features are extracted from the at least one of 1) the one or more input medical images or 2) the input text-based medical data using at least one of a machine learning based image encoder or a machine learning based text encoder. One or more medical imaging analysis tasks are performed based on the extracted features using one or more machine learning based decoders. Results of the one or more medical imaging analysis tasks are output. The at least one of the machine learning based image encoder or the machine learning based text encoder are trained by receiving image/text pairs each comprising a training medical image and training text-based medical data, encoding the training medical images into image features using the machine learning based image encoder, encoding the training text-based medical data into text features using the machine learning based text encoder, extracting clinical labels from the training text-based medical data using one or more language models, and training the at least one of the machine learning based image encoder or the machine learning based text encoder based on the image features, the text features, and the clinical labels.

In one embodiment, a clinical context loss is computed by clustering the image features and the text features of each of the image/text pairs based on the clinical labels. In another embodiment, a contrastive learning loss is computed by minimizing a distance between the image features and the text features from the same image/text pair and maximizing distances between the image features and the text features from different ones of the image/text pairs. The clinical context loss and the contrastive learning loss may be jointly optimized.

In one embodiment, clinically relevant sentences are extracted from the training text-based medical data using a first language model. The clinical labels are determined for the clinically relevant sentences using a second language model.

In accordance with one or more embodiments, systems and methods for training a medical foundation model are provided. Image/text pairs each comprising a training medical image and training text-based medical data are received. The training medical images are encoded into image features using a machine learning based image encoder. The training text-based medical data are encoded into text features using a machine learning based text encoder. Clinical labels are extracted from the training text-based medical data using one or more language models. At least one of the machine learning based image encoder or the machine learning based text encoder are trained based on the image features, the text features, and the clinical labels.

These and other advantages of the invention will be apparent to those of ordinary skill in the art by reference to the following detailed description and the accompanying drawings.

### BRIEF DESCRIPTION OF THE DRAWINGS

FIG. 1 shows a method for training a medical foundation model, in accordance with one or more embodiments;

FIG. 2 shows a workflow for training a medical foundation model, in accordance with one or more embodiments;

FIG. 3 shows a method for performing one or more medical imaging analysis task, in accordance with one or more embodiments;

FIG. 4 shows an exemplary artificial neural network that may be used to implement one or more embodiments;

FIG. 5 shows a convolutional neural network that may be used to implement one or more embodiments;

FIG. 6 shows a schematic structure of a recurrent machine learning model that may be used to implement one or more embodiments; and

FIG. 7 shows a high-level block diagram of a computer that may be used to implement one or more embodiments.

### DETAILED DESCRIPTION

The present invention generally relates to methods and systems for multi-finding abnormality detection using a medical AI (artificial intelligence) foundation model. Embodiments of the present invention are described herein to give a visual understanding of such methods and systems. A digital image is often composed of digital representations of one or more objects (or shapes). The digital representation of an object is often described herein in terms of identifying and manipulating the objects. Such manipulations are virtual manipulations accomplished in the memory or other circuitry/hardware of a computer system. Accordingly, is to be understood that embodiments of the present invention may be performed within a computer system using data stored within the computer system. Further, reference herein to pixels of an image may refer equally to voxels of an image and vice versa.

Embodiments described herein provide for a framework for training a medical foundation model to learn clinically representative features for effective and comprehensive detection of multiple abnormalities in medical images. The framework employs a joint optimization of a contrastive loss with an LLM (large language model)-based clustering loss for training the medical foundation model. The LLM-based clustering loss clusters features extracted from training medical images and training text-based medical data around clinical labels extracted from the training text-based medical data by an LLM. Advantageously, the training of the medical foundation model by the joint optimization results in the performance of downstream medical imaging analysis tasks with increased performance and accuracy as compared with conventionally trained medical foundation models.

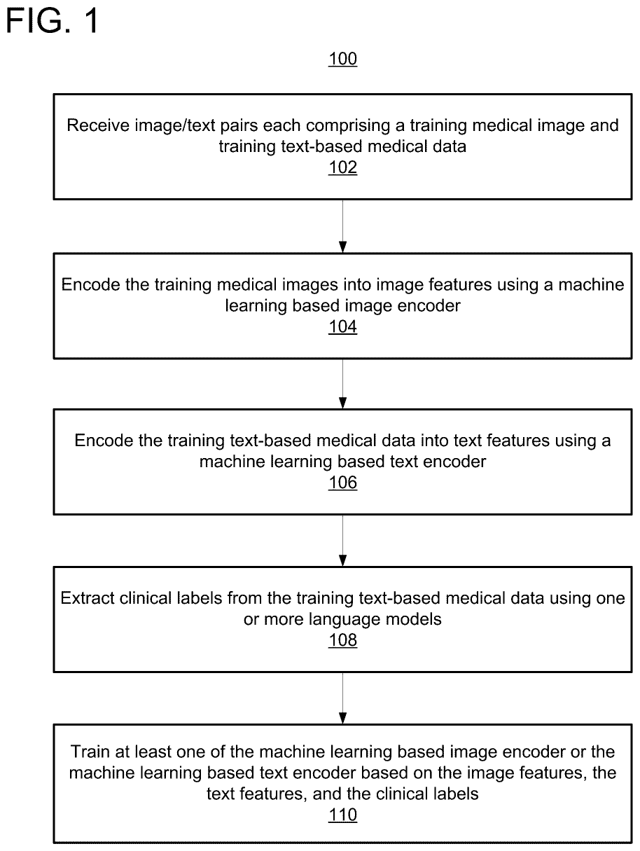

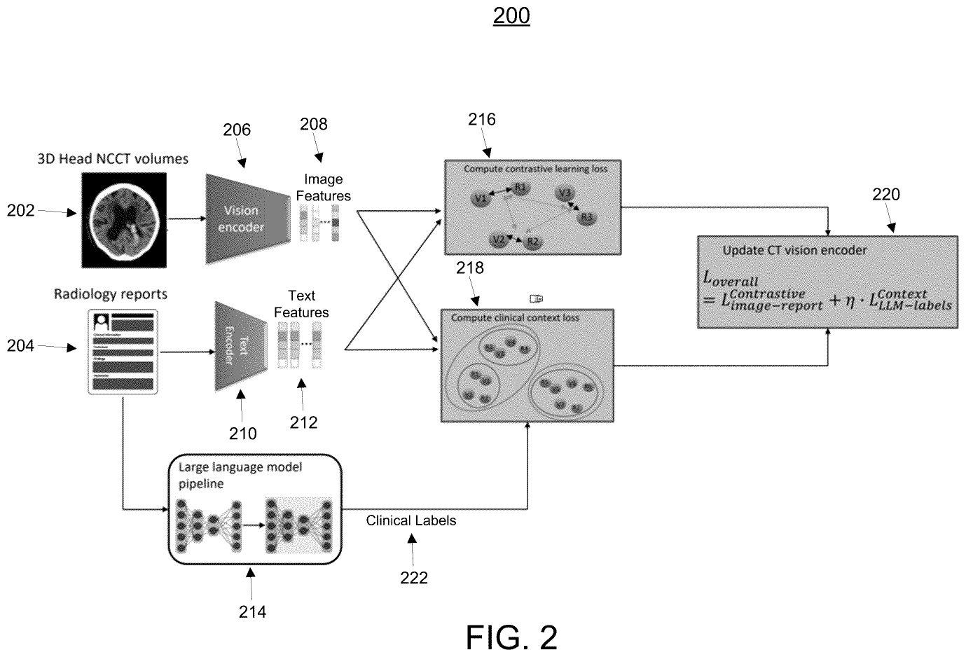

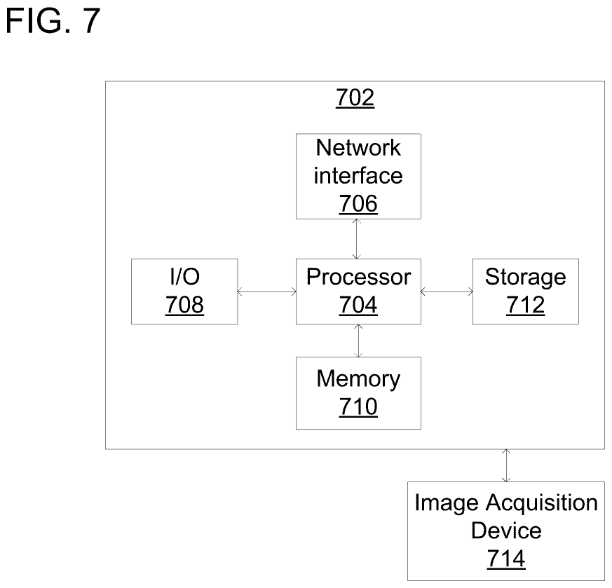

FIG. 1 shows a method 100 for training a medical foundation model, in accordance with one or more embodiments. The steps and sub-steps of method 100 may be performed by one or more suitable computing devices, such as, e.g., computer 702 of FIG. 7. FIG. 2 shows a workflow 200 for training a medical foundation model, in accordance with one or more embodiments. FIG. 1 and FIG. 2 will be described together.

At step 102 of FIG. 1, image/text pairs each comprising a training medical image and training text-based medical data are received. For each image/text pair, the training medical image and the training text-based medical data relate to a same anatomical object of interest (e.g., organ, bone, vessel, tumor or other abnormality, etc.). In one example, as shown in workflow 200 of FIG. 2, the training medical image may be 3D head NCCT (non-contrast computed tomography) volumes 202 and the training text-based medical data may be radiology reports 204.

The training medical images may be of any suitable modality, such as, e.g., MRI (magnetic resonance imaging), CT (computed tomography), US (ultrasound), x-ray, or any other medical imaging modality or combinations of medical imaging modalities. The training medical images may comprise 2D (two dimensional) images and/or 3D (three dimensional) volumes, and each image/text pair may comprise any number of training medical images.

The training text-based medical data may comprise any text-based medical data. In one example, the training text-based medical data comprises radiology reports of a patient. However, the training text-based medical data may comprise any other suitable text-based data of a patient, such as, e.g., other types of reports (e.g., clinical reports), demographic information, vital signs, medical history, family history, laboratory results, measurements and information extracted from medical images, etc. of a patient.

The image/text pairs may be received, for example, by directly receiving medical images from an image acquisition device (e.g., image acquisition device 714 of FIG. 7) as the training medical images are acquired, by loading the image/text pairs from a storage or memory of a computer system (e.g., storage 712 or memory 710 of computer 702 of FIG. 7), and/or by receiving the image/text pairs from a remote computer system (e.g., computer 702 of FIG. 7). Such a computer system or remote computer system may comprise one or more patient databases, such as, e.g., an HER (electronic health record), EMR (electronic medical record), PHR (personal health record), HIS (health information system), RIS (radiology information system), PACS (picture archiving and communication system), LIMS (laboratory information management system), or any other suitable database or system.

At step 104 of FIG. 1, the training medical images are encoded into image features using a machine learning based image encoder. In one example, as shown in workflow 200 of FIG. 2, volumes 202 are encoded into images features 208 by vision encoder 206.

In one embodiment, the machine learning based image encoder is a ViT (vision transformer). However, the machine learning based image encoder may be implemented according to any other suitable machine learning based architecture. The machine learning based image encoder extracts the image features from the training medical images by dividing the training medical images into smaller patches and processing the patches through the machine learning based image encoder. Accordingly, the machine learning based image encoder receives as input the training medical images (or patches extracted therefrom) and generates as output the images features. The image features are low-level latent representations or embeddings of the training medical images in a vector representation. The image features may comprise, for example, local features (e.g., edges, textures, simple shapes) of the training medical images, global context (e.g., relationships between distant patches) of the training medical images, object relationships (e.g., relationships between objects, parts, and spatial layouts) of the training medical images, and high-level semantic features (e.g., higher-level concepts such as objects, scenes, and their interactions) of the training medical images.

At step 106 of FIG. 1, the training text-based medical data is encoded into text features using a machine learning based text encoder. In one example, as shown in workflow 200 of FIG. 2, radiology reports 204 are encoded into text features 212 using text encoder 210. The machine learning based image encoder and the machine learning based text encoder together form the medical foundation model.

In one embodiment, the machine learning based text encoder is a transformer-based text encoder, such as, e.g., GPT (generative pre-training transformer), BLOOM (BigScience Large Open-science Open-access Multilingual Language Model), or BERT (bi-directional encoder representations from transformers). However, the machine learning based text encoder may be implemented according to any other suitable machine learning based architecture. The machine learning based text encoder extracts the text features from the training text-based medical data by dividing the training text-based medical data into tokens and mapping each token to a dense vector. Accordingly, the machine learning based text encoder receives as input the training text-based medical data (or tokens extracted therefrom) and generates as output the text features. The text features are low-level latent representations or embeddings of the training text-based medical data in a vector representation.

At step 108 of FIG. 1, clinical labels are extracted from the training text-based medical data using one or more language models. In one example, as shown in workflow 200 of FIG. 2, clinical labels 222 are extracted from radiology reports 204 by large language model pipeline 214.

In one embodiment, the language model is an LLM (large language model). The LLM may be any suitable pretrained deep learning based LLM. For example, the LLM may be based on the transformer architecture, which uses an attention mechanism to capture long-range dependencies in text. One example of a transformer-based architecture is GPT, which has a multilayer transformer decoder architecture that may be pretrained to optimize the next token prediction task and then fine-tuned with labelled data for various downstream tasks. Other exemplary transformer-based architectures include BLOOM and BERT. However, the language model may be any other suitable language model. For example, the language model may be a small language model, which uses a relatively smaller neural network, has fewer parameters, and is trained on less training data as compared with an LLM. The language model receives as input the training text-based medical data (and instructions) and generates as output the clinical labels.

In one embodiment, the one or more language models comprise a search language model and a clinical decision language model. The search language model extracts clinically relevant sentences from the training text-based medical data. The clinical decision language model determines a case-level clinical label for each target finding in the clinically relevant sentences. In one embodiment, the clinical labels may be extracted from the training text-based medical data during an offline preprocessing stage.

At step 110 of FIG. 1, at least one of the machine learning based image encoder or the machine learning based text encoder are trained based on the image features, the text features, and the clinical labels. In one embodiment, at least one of the machine learning based image encoder or the machine learning based text encoder are trained by jointly optimizing a contrastive learning loss and a clinical context loss. The joint optimization provides for a clinical context-guided contrastive representation learning performed in the latent space with features of the training medical images and the training text-based medical data with the clinical labels. In one example, as shown in workflow 200 of FIG. 2, a contrastive learning loss 216 is computed based on image features 208 and text features 212 and a clinical context loss 218 is computed based on image features 208, text features 212, and clinical labels 222 and vision encoder 206 is updated 220 according to an overall loss function based on the contrastive learning loss and the clinical loss.

The contrastive learning loss

$${ℒ}_{image - text}^{contrastive}$$

aims to minimize the distances between the image features and the text features from the same image/text pair while maximizing the distances between the image features and the text features from different ones of the image/text pairs by employing distance losses such as, e.g., normalized temperature scaled cross entropy loss or cosine similarity loss for contrastive training. The clinical context loss

$${ℒ}_{c linical labels}^{context}$$

aims to cluster the image features and the text features of each of the image/text pairs having a same or similar clinical context based on the clinical labels. The overall training object is to optimize the exemplary loss function of Equation (1) in an unsupervised manner:

$${ℒ}_{overall} = {ℒ}_{image - text}^{contrastive} + η \cdot {ℒ}_{clinical labels}^{context} \qquad ( 1 )$$

The contrastive learning loss

$${ℒ}_{image - text}^{contrastive}$$

ensures similarity between features of image/text pairs while maintaining dissimilarity for features of unpaired images/text. Normalized temperature scaled cross entropy loss or cosine similarity based contrastive loss can be used as the contrastive learning loss

$${ℒ}_{image - text}^{contrastive} .$$

The clinical context loss

$${ℒ}_{c linical labels}^{context}$$

focuses on high-level clinical concepts, clustering feature distances of image/text pairs from the same semantic categories using the clinical labels. The KL (Kullback-Leibler) divergence can be used to push the cluster assignment probability towards the target distribution using soft cluster frequency approximation within a mini-batch during model training. The clinical context-based clustering can be performed with hierarchical clustering to build a hierarchy of each clinical context. More specifically, each data point (i.e., features of an image-report pair) is considered as a single cluster and the pairwise distance between all data points is calculated to form the distance matrix. Based on the distance matrix, the pairs of clusters that are closest to each other are identified, and then the identified pair of clusters is merged into a single cluster. The distance matrix is recalculated between the new clusters and all other clusters using several methods, such as, e.g., single/complete linkage, average linkage, or Ward's method. The hierarchical clustering process is iterated until the distance between clusters does not exceed a certain threshold. η is a hyper-parameter controlling the balance between contrastive learning and clustering during the training.

Method 100 may be repeated for one or more iterations and/or one or more epoch for training the at least one of the machine learning based image encoder or the machine learning based text encoder. Once trained, the at least one of the machine learning based image encoder or the machine learning based text encoder may be stored on a memory or storage of a computer system (e.g., memory 710 or storage 712 of computer 702 of FIG. 7) or transmitted to a remote computer system (e.g., computer 702 of FIG. 7).

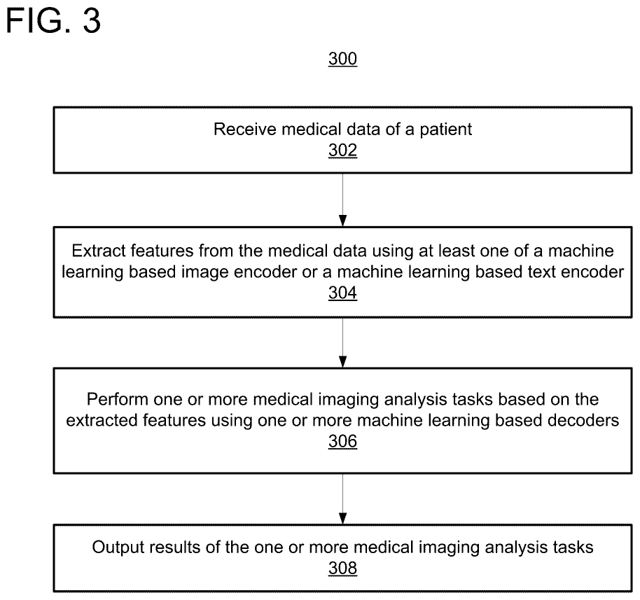

In one embodiment, the trained at least one of the machine learning based image encoder or the machine learning based text encoder may be applied during an online or inference stage, e.g., to perform method 300 of FIG. 3. The trained at least one of the machine learning based image encoder or the machine learning based text encoder may first be fine-tuned with specific multi-finding downstream tasks prior to be applied.

FIG. 3 shows a method 300 for performing one or more medical imaging analysis task, in accordance with one or more embodiments. The steps and sub-steps of method 300 may be performed by one or more suitable computing devices, such as, e.g., computer 702 of FIG. 7.

At step 302 of FIG. 2, medical data of a patient is received. The medical data may comprise at least one of 1) one or more input medical images or 2) input text-based medical data. The one or more input medical images may be of any suitable modality, and may comprise 2D images and/or 3D volumes. The input text-based medical data may comprise radiology reports or any other suitable text-based data of a patient. The medical data may be received, for example, by directly receiving the medical data (i.e., the one or more input medical images) from an image acquisition device (e.g., image acquisition device 714 of FIG. 7) as the images are acquired, by loading the medical data from a storage or memory of a computer system (e.g., storage 712 or memory 710 of computer 702 of FIG. 7), and/or by receiving the medical data from a remote computer system (e.g., computer 702 of FIG. 7).

At step 304 of FIG. 3, features are extracted from the medical data using at least one of a machine learning based image encoder or a machine learning based text encoder. The at least one of the machine learning based image encoder or the machine learning based text encoder are trained according to method 100 of FIG. 1. The machine learning based image encoder and/or a machine learning based text encoder respectively receive as input the medical data and generates as output the extracted features (i.e., image features and/or text features).

At step 306 of FIG. 3, one or more medical imaging analysis tasks are performed based on the extracted features using one or more machine learning based decoders. The one or more medical imaging analysis task may comprise, for example, registration, segmentation, classification, detection, or any other suitable medical imaging analysis task. In one embodiment, the one or more machine learning based decoders comprise transformer-based decoders. However, the one or more machine learning based decoders may be implemented according to any other machine learning based architecture. The one or more machine learning based decoders receive as input the extracted features and decode the extracted features to respectively generate as output results of the one or more medical imaging analysis task.

At step 308 of FIG. 3, results of the one or more medical imaging analysis tasks are output. For example, the results of the one or more medical imaging analysis tasks can be output by displaying the results on a display device of a computer system (e.g., I/O 708 of computer 702 of FIG. 7), storing the results on a memory or storage of a computer system (e.g., memory 710 or storage 712 of computer 702 of FIG. 7), or by transmitting the results to a remote computer system (e.g., computer 702 of FIG. 7).

Advantageously, embodiments disclosed herein provides for unsupervised pretraining of a medical foundation model by learning clinically representation features. It achieves this by jointly optimizing a contrastive loss with an LLM-based clustering loss. The joint optimization facilitates clinical context-aware contrastive learning, leading to improved performance in downstream medical imaging analysis tasks. Consequently, embodiments described herein can be used for effective and comprehensive detection of multiple abnormalities from medical images. Further, embodiments described herein may be effectively scaled with large datasets and a wide variety of clinical findings without the need for manual expert intervention. In addition, the medical foundation model in accordance with embodiments described herein may be fine-tuned for automatically analyzing medical images, such as, e.g., head CT scans, to identify multiple neurological conditions and generate comprehensive diagnostic reports. Embodiments described herein may assist radiologists by providing quick and accurate preliminary diagnoses, thereby enhancing the efficiency and accuracy of neurological condition detection.

Embodiments described herein are described with respect to the claimed systems as well as with respect to the claimed methods. Features, advantages or alternative embodiments herein can be assigned to the other claimed objects and vice versa. In other words, claims and embodiments for the systems can be improved with features described or claimed in the context of the respective methods. In this case, the functional features of the method are implemented by physical units of the system.

Furthermore, certain embodiments described herein are described with respect to methods and systems utilizing trained machine learning models, as well as with respect to methods and systems for providing trained machine learning models. Features, advantages or alternative embodiments herein can be assigned to the other claimed objects and vice versa. In other words, claims and embodiments for providing trained machine learning models can be improved with features described or claimed in the context of utilizing trained machine learning models, and vice versa. In particular, datasets used in the methods and systems for utilizing trained machine learning models can have the same properties and features as the corresponding datasets used in the methods and systems for providing trained machine learning models, and the trained machine learning models provided by the respective methods and systems can be used in the methods and systems for utilizing the trained machine learning models.

In general, a trained machine learning model mimics cognitive functions that humans associate with other human minds. In particular, by training based on training data the machine learning model is able to adapt to new circumstances and to detect and extrapolate patterns. Another term for “trained machine learning model” is “trained function.”

In general, parameters of a machine learning model can be adapted by means of training. In particular, supervised training, semi-supervised training, unsupervised training, reinforcement learning and/or active learning can be used. Furthermore, representation learning (an alternative term is “feature learning”) can be used. In particular, the parameters of the machine learning models can be adapted iteratively by several steps of training. In particular, within the training a certain cost function can be minimized. In particular, within the training of a neural network the backpropagation algorithm can be used.

In particular, a machine learning model, such as, e.g., the machine learning based image encoder utilized at step 104, the machine learning based text encoder utilized at step 106, and the one or more language models utilized at step 108 of FIG. 1, vision encoder 206, text encoder 210, large language model pipeline 214 of FIG. 2, and the machine learning based image encoder and the machine learning based text encoder utilized at step 304 and the one or more machine learning based decoders utilized at step 306 of FIG. 3, can comprise, for example, a neural network, a support vector machine, a decision tree and/or a Bayesian network, and/or the machine learning model can be based on, for example, k-means clustering, Q-learning, genetic algorithms and/or association rules. In particular, a neural network can be, e.g., a deep neural network, a convolutional neural network or a convolutional deep neural network. Furthermore, a neural network can be, e.g., an adversarial network, a deep adversarial network and/or a generative adversarial network.

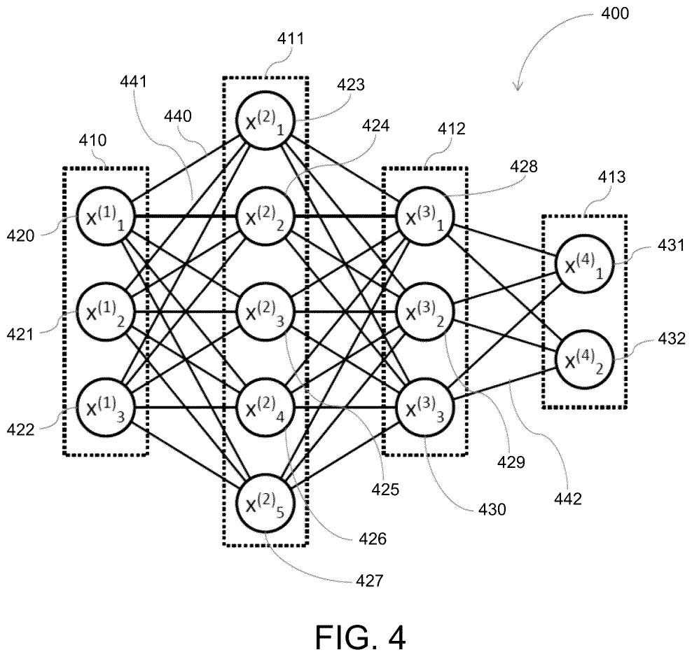

FIG. 4 shows an embodiment of an artificial neural network 400 that may be used to implement one or more machine learning models described herein. Alternative terms for “artificial neural network” are “neural network”, “artificial neural net” or “neural net”.

The artificial neural network 400 comprises nodes 420, . . . , 432 and edges 440, . . . , 442, wherein each edge 440, . . . , 442 is a directed connection from a first node 420, . . . , 432 to a second node 420, . . . , 432. In general, the first node 420, . . . , 432 and the second node 420, . . . , 432 are different nodes 420, . . . , 432, it is also possible that the first node 420, . . . , 432 and the second node 420, . . . , 432 are identical. For example, in FIG. 4 the edge 440 is a directed connection from the node 420 to the node 423, and the edge 442 is a directed connection from the node 430 to the node 432. An edge 440, . . . , 442 from a first node 420, . . . , 432 to a second node 420, . . . , 432 is also denoted as “ingoing edge” for the second node 420, . . . , 432 and as “outgoing edge” for the first node 420, . . . , 432.

In this embodiment, the nodes 420, . . . , 432 of the artificial neural network 400 can be arranged in layers 410, . . . , 413, wherein the layers can comprise an intrinsic order introduced by the edges 440, . . . , 442 between the nodes 420, . . . , 432. In particular, edges 440, . . . , 442 can exist only between neighboring layers of nodes. In the displayed embodiment, there is an input layer 410 comprising only nodes 420, . . . , 422 without an incoming edge, an output layer 413 comprising only nodes 431, 432 without outgoing edges, and hidden layers 411, 412 in-between the input layer 410 and the output layer 413. In general, the number of hidden layers 411, 412 can be chosen arbitrarily. The number of nodes 420, . . . , 422 within the input layer 410 usually relates to the number of input values of the neural network, and the number of nodes 431, 432 within the output layer 413 usually relates to the number of output values of the neural network.

In particular, a (real) number can be assigned as a value to every node 420, . . . , . . . , 432 of the neural network 400. Here, x⁽ⁿ⁾ᵢ denotes the value of the i-th node 420, . . . , 432 of the n-th layer 410, . . . , 413. The values of the nodes 420, . . . , 422 of the input layer 410 are equivalent to the input values of the neural network 400, the values of the nodes 431, 432 of the output layer 413 are equivalent to the output value of the neural network 400. Furthermore, each edge 440, . . . , 442 can comprise a weight being a real number, in particular, the weight is a real number within the interval [−1, 1] or within the interval [0, 1]. Here, w^{(m,n)}_{i,j} denotes the weight of the edge between the i-th node 420, . . . , 432 of the m-th layer 410, . . . , 413 and the j-th node 420, . . . , 432 of the n-th layer 410, . . . , 413. Furthermore, the abbreviation w⁽ⁿ⁾_{i,j} is defined for the weight w^{(n,n+1)}_{i,j}.

In particular, to calculate the output values of the neural network 400, the input values are propagated through the neural network. In particular, the values of the nodes 420, . . . , 432 of the (n+1)-th layer 410, . . . , 413 can be calculated based on the values of the nodes 420, . . . , 432 of the n-th layer 410, . . . , 413 by

$${x}^{{( n + 1 )}_{j}} = f ( {\sum }_{i} {x}^{{( n )}_{i}} \cdot {w}^{{( n )}_{i , j}} ) .$$

Herein, the function f is a transfer function (another term is “activation function”). Known transfer functions are step functions, sigmoid function (e.g., the logistic function, the generalized logistic function, the hyperbolic tangent, the Arctangent function, the error function, the smoothstep function) or rectifier functions. The transfer function is mainly used for normalization purposes.

In particular, the values are propagated layer-wise through the neural network, wherein values of the input layer 410 are given by the input of the neural network 400, wherein values of the first hid-den layer 411 can be calculated based on the values of the input layer 410 of the neural network, wherein values of the second hidden layer 412 can be calculated based in the values of the first hidden layer 411, etc.

In order to set the values w^{(m,n)}_{i,j} for the edges, the neural network 400 has to be trained using training data. In particular, training data comprises training input data and training output data (denoted as tᵢ). For a training step, the neural network 400 is applied to the training input data to generate calculated output data. In particular, the training data and the calculated output data comprise a number of values, said number being equal with the number of nodes of the output layer.

In particular, a comparison between the calculated output data and the training data is used to recursively adapt the weights within the neural network 400 (backpropagation algorithm). In particular, the weights are changed according to

w′⁽ⁿ⁾^{_{i,j}}=w⁽ⁿ⁾^{_{i,j}}−γ·δ⁽ⁿ⁾^{ⱼ}·x⁽ⁿ⁾^{ᵢ}

wherein γ is a learning rate, and the numbers δ⁽ⁿ⁾ⱼ can be recursively calculated as

$${δ}^{{( n )}_{j}} = ( {\sum }_{k} {δ}^{{( n + 1 )}_{k}} \cdot {w}^{{( n + 1 )}_{j , k}} ) \cdot {f}^{′} ( {\sum }_{i} {x}^{{( n )}_{i}} \cdot {w}^{{( n )}_{i , j}} )$$

based on δ⁽ⁿ⁺¹⁾ⱼ, if the (n+1)-th layer is not the output layer, and

$${δ}^{{( n )}_{j}} = ( {x}^{{( n + 1 )}_{j}} - {t}^{{( n + 1 )}_{j}} ) \cdot {f}^{′} ( {x}^{{( n )}_{i}} \cdot {w}^{{( n )}_{i , j}} )$$

if the (n+1)-th layer is the output layer 413, wherein f′ is the first derivative of the activation function, and t⁽ⁿ⁺¹⁾ⱼ is the comparison training value for the j-th node of the output layer 413.

A convolutional neural network is a neural network that uses a convolution operation instead of general matrix multiplication in at least one of its layers (so-called “convolutional layer”). In particular, a convolutional layer performs a dot product of one or more convolution kernels with the convolutional layer's input data/image, wherein the entries of the one or more convolution kernels are the parameters or weights that are adapted by training. In particular, one can use the Frobenius inner product and the ReLU activation function. A convolutional neural network can comprise additional layers, e.g., pooling layers, fully connected layers, and normalization layers.

By using convolutional neural networks input images can be processed in a very efficient way, because a convolution operation based on different kernels can extract various image features, so that by adapting the weights of the convolution kernel the relevant image features can be found during training. Furthermore, based on the weight-sharing in the convolutional kernels less parameters need to be trained, which prevents overfitting in the training phase and allows to have faster training or more layers in the network, improving the performance of the network.

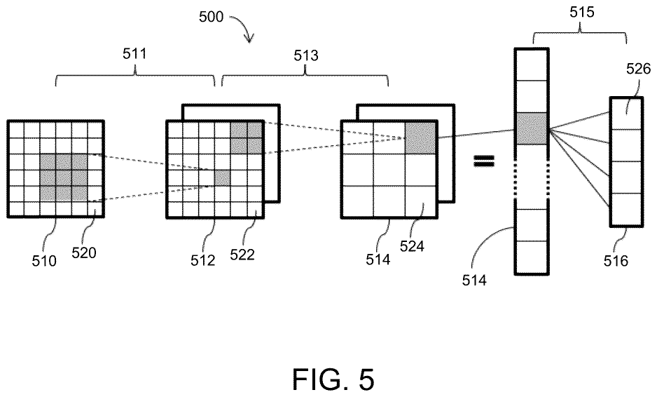

FIG. 5 shows an embodiment of a convolutional neural network 500 that may be used to implement one or more machine learning models described herein. In the displayed embodiment, the convolutional neural network 500 comprises an input node layer 510, a convolutional layer 511, a pooling layer 513, a fully connected layer 514 and an output node layer 516, as well as hidden node layers 512, 514. Alternatively, the convolutional neural network 500 can comprise several convolutional layers 511, several pooling layers 513 and several fully connected layers 515, as well as other types of layers. The order of the layers can be chosen arbitrarily, usually fully connected layers 515 are used as the last layers before the output layer 516.

In particular, within a convolutional neural network 500 nodes 520, 522, 524 of a node layer 510, 512, 514 can be considered to be arranged as a d-dimensional matrix or as a d-dimensional image. In particular, in the two-dimensional case the value of the node 520, 522, 524 indexed with i and j in the n-th node layer 510, 512, 514 can be denoted as x(n)[i, j]. However, the arrangement of the nodes 520, 522, 524 of one node layer 510, 512, 514 does not have an effect on the calculations executed within the convolutional neural network 500 as such, since these are given solely by the structure and the weights of the edges.

A convolutional layer 511 is a connection layer between an anterior node layer 510 (with node values x(n−1)) and a posterior node layer 512 (with node values x(n)). In particular, a convolutional layer 511 is characterized by the structure and the weights of the incoming edges forming a convolution operation based on a certain number of kernels. In particular, the structure and the weights of the edges of the convolutional layer 511 are chosen such that the values x(n) of the nodes 522 of the posterior node layer 512 are calculated as a convolution x(n)=K*x(n−1) based on the values x(n−1) of the nodes 520 anterior node layer 510, where the convolution * is defined in the two-dimensional case as

$${x}_{k}^{( n )} [ i , j ] = ( K * {x}^{( n - 1 )} ) [ i , j ] = {\sum }_{{i}^{′}} {\sum }_{{j}^{′}} K [ {i}^{′} , {j}^{′} ] \cdot {x}^{( n - 1 )} [ i - {i}^{′} , j - {j}^{′} ] .$$

Here the kernel K is a d-dimensional matrix (in this embodiment, a two-dimensional matrix), which is usually small compared to the number of nodes 520, 522 (e.g., a 3×3 matrix, or a 5×5 matrix). In particular, this implies that the weights of the edges in the convolution layer 511 are not independent, but chosen such that they produce said convolution equation. In particular, for a kernel being a 3×3 matrix, there are only 9 independent weights (each entry of the kernel matrix corresponding to one independent weight), irrespectively of the number of nodes 520, 522 in the anterior node layer 510 and the posterior node layer 512.

In general, convolutional neural networks 500 use node layers 510, 512, 514 with a plurality of channels, in particular, due to the use of a plurality of kernels in convolutional layers 511. In those cases, the node layers can be considered as (d+1)-dimensional matrices (the first dimension indexing the channels). The action of a convolutional layer 511 is then a two-dimensional example defined as

$${x}^{{( n )}_{b}} [ i , j ] = {\sum }_{a} {K}_{a , b} * {x}^{{( n - 1 )}_{a}} [ i , j ] = {\sum }_{a} {\sum }_{{i}^{′}} {\sum }_{{j}^{′}} {K}_{a , b} [ {i}^{′} , {j}^{′} ] \cdot {x}^{{( n - 1 )}_{a}} [ i - {i}^{′} , j - {j}^{′} ]$$

where x^{(n-1)a} corresponds to the a-th channel of the anterior node layer 510, x⁽ⁿ⁾_{b} corresponds to the b-th channel of the posterior node layer 512 and K_{a,b} corresponds to one of the kernels. If a convolutional layer 511 acts on an anterior node layer 510 with A channels and outputs a posterior node layer 512 with B channels, there are A·B independent d-dimensional kernels K_{a,b}.

In general, in convolutional neural networks 500 activation functions are used. In this embodiment ReLU (acronym for “Rectified Linear Units”) is used, with R(z)=max(0, z), so that the action of the convolutional layer 511 in the two-dimensional example is

$${x}^{{( n )}_{b}} [ i , j ] = R ( {\sum }_{a} ( {K}_{a , b} * {x}^{{( n - 1 )}_{a}} ) [ i , j ] ) = R ( {\sum }_{a} {\sum }_{{i}^{′}} {\sum }_{{j}^{′}} {K}_{a , b} [ {i}^{′} , {j}^{′} ] \cdot {x}^{{( n - 1 )}_{a}} [ i - {i}^{′} , j - {j}^{′} ] )$$

It is also possible to use other activation functions, e.g., ELU (acronym for “Exponential Linear Unit”), LeakyReLU, Sigmoid, Tanh or Softmax.

In the displayed embodiment, the input layer 510 comprises 36 nodes 520, arranged as a two-dimensional 6×6 matrix. The first hidden node layer 512 comprises 72 nodes 522, arranged as two two-dimensional 6×6 matrices, each of the two matrices being the result of a convolution of the values of the input layer with a 3×3 kernel within the convolutional layer 511. Equivalently, the nodes 522 of the first hidden node layer 512 can be interpreted as arranged as a three-dimensional 2×6×6 matrix, wherein the first dimension correspond to the channel dimension.

The advantage of using convolutional layers 511 is that spatially local correlation of the input data can exploited by enforcing a local connectivity pattern between nodes of adjacent layers, in particular by each node being connected to only a small region of the nodes of the preceding layer.

A pooling layer 513 is a connection layer between an anterior node layer 512 (with node values x(n−1)) and a posterior node layer 514 (with node values x(n)). In particular, a pooling layer 513 can be characterized by the structure and the weights of the edges and the activation function forming a pooling operation based on a non-linear pooling function f. For example, in the two-dimensional case the values x(n) of the nodes 524 of the posterior node layer 514 can be calculated based on the values x(n−1) of the nodes 522 of the anterior node layer 512 as

$${x}^{{( n )}_{b}} [ i , j ] = f \{ {x}^{( n - 1 )} [ {id}_{1} , {jd}_{2} ] , … , {x}^{{( n - 1 )}_{b}} [ ( i + 1 ) {d}_{1} - 1 , ( j + 1 ) {d}_{2} - 1 ] )$$

In other words, by using a pooling layer 513 the number of nodes 522, 524 can be reduced, by re-placing a number d1·d2 of neighboring nodes 522 in the anterior node layer 512 with a single node 522 in the posterior node layer 514 being calculated as a function of the values of said number of neighboring nodes. In particular, the pooling function f can be the max-function, the average or the L2-Norm. In particular, for a pooling layer 513 the weights of the incoming edges are fixed and are not modified by training.

The advantage of using a pooling layer 513 is that the number of nodes 522, 524 and the number of parameters is reduced. This leads to the amount of computation in the network being reduced and to a control of overfitting.

In the displayed embodiment, the pooling layer 513 is a max-pooling layer, replacing four neighboring nodes with only one node, the value being the maximum of the values of the four neighboring nodes. The max-pooling is applied to each d-dimensional matrix of the previous layer; in this embodiment, the max-pooling is applied to each of the two two-dimensional matrices, reducing the number of nodes from 72 to 18.

In general, the last layers of a convolutional neural network 500 are fully connected layers 515. A fully connected layer 515 is a connection layer between an anterior node layer 514 and a posterior node layer 516. A fully connected layer 513 can be characterized by the fact that a majority, in particular, all edges between nodes 514 of the anterior node layer 514 and the nodes 516 of the posterior node layer are present, and wherein the weight of each of these edges can be adjusted individually.

In this embodiment, the nodes 524 of the anterior node layer 514 of the fully connected layer 515 are displayed both as two-dimensional matrices, and additionally as non-related nodes (indicated as a line of nodes, wherein the number of nodes was reduced for a better presentability). This operation is also denoted as “flattening”. In this embodiment, the number of nodes 526 in the posterior node layer 516 of the fully connected layer 515 smaller than the number of nodes 524 in the anterior node layer 514. Alternatively, the number of nodes 526 can be equal or larger.

Furthermore, in this embodiment the Softmax activation function is used within the fully connected layer 515. By applying the Softmax function, the sum the values of all nodes 526 of the output layer 516 is 1, and all values of all nodes 526 of the output layer 516 are real numbers between 0 and 1. In particular, if using the convolutional neural network 500 for categorizing input data, the values of the output layer 516 can be interpreted as the probability of the input data falling into one of the different categories.

In particular, convolutional neural networks 500 can be trained based on the backpropagation algorithm. For preventing overfitting, methods of regularization can be used, e.g., dropout of nodes 520, . . . , 524, stochastic pooling, use of artificial data, weight decay based on the L1 or the L2 norm, or max norm constraints.

According to an aspect, the machine learning model may comprise one or more residual networks (ResNet). In particular, a ResNet is an artificial neural network comprising at least one jump or skip connection used to jump over at least one layer of the artificial neural network. In particular, a ResNet may be a convolutional neural network comprising one or more skip connections respectively skipping one or more convolutional layers. According to some examples, the ResNets may be represented as m-layer ResNets, where m is the number of layers in the corresponding architecture and, according to some examples, may take values of 34, 50, 101, or 152. According to some examples, such an m-layer ResNet may respectively comprise (m−2)/2 skip connections.

A skip connection may be seen as a bypass which directly feeds the output of one preceding layer over one or more bypassed layers to a layer succeeding the one or more bypassed layers. Instead of having to directly fit a desired mapping, the bypassed layers would then have to fit a residual mapping “balancing” the directly fed output.

Fitting the residual mapping is computationally easier to optimize than the directed mapping. What is more, this alleviates the problem of vanishing/exploding gradients during optimization upon training the machine learning models: if a bypassed layer runs into such problems, its contribution may be skipped by regularization of the directly fed output. Using ResNets thus brings about the advantage that much deeper networks may be trained.

In particular, a recurrent machine learning model is a machine learning model whose output does not only depend on the input value and the parameters of the machine learning model adapted by the training process, but also on a hidden state vector, wherein the hidden state vector is based on previous inputs used on for the recurrent machine learning model. In particular, the recurrent machine learning model can comprise additional storage states or additional structures that incorporate time delays or comprise feedback loops.

In particular, the underlying structure of a recurrent machine learning model can be a neural network, which can be denoted as recurrent neural network. Such a recurrent neural network can be described as an artificial neural network where connections between nodes form a directed graph along a temporal sequence. In particular, a recurrent neural network can be interpreted as directed acyclic graph. In particular, the recurrent neural network can be a finite impulse recurrent neural network or an infinite impulse recurrent neural network (wherein a finite impulse network can be unrolled and replaced with a strictly feedforward neural network, and an infinite impulse network cannot be unrolled and replaced with a strictly feedforward neural network).

In particular, training a recurrent neural network can be based on the BPTT algorithm (acronym for “backpropagation through time”), on the RTRL algorithm (acronym for “real-time recurrent learning”) and/or on genetic algorithms.

By using a recurrent machine learning model input data comprising sequences of variable length can be used. In particular, this implies that the method cannot be used only for a fixed number of input datasets (and needs to be trained differently for every other number of input datasets used as input), but can be used for an arbitrary number of input datasets. This implies that the whole set of training data, independent of the number of input datasets contained in different sequences, can be used within the training, and that training data is not reduced to training data corresponding to a certain number of successive input datasets.

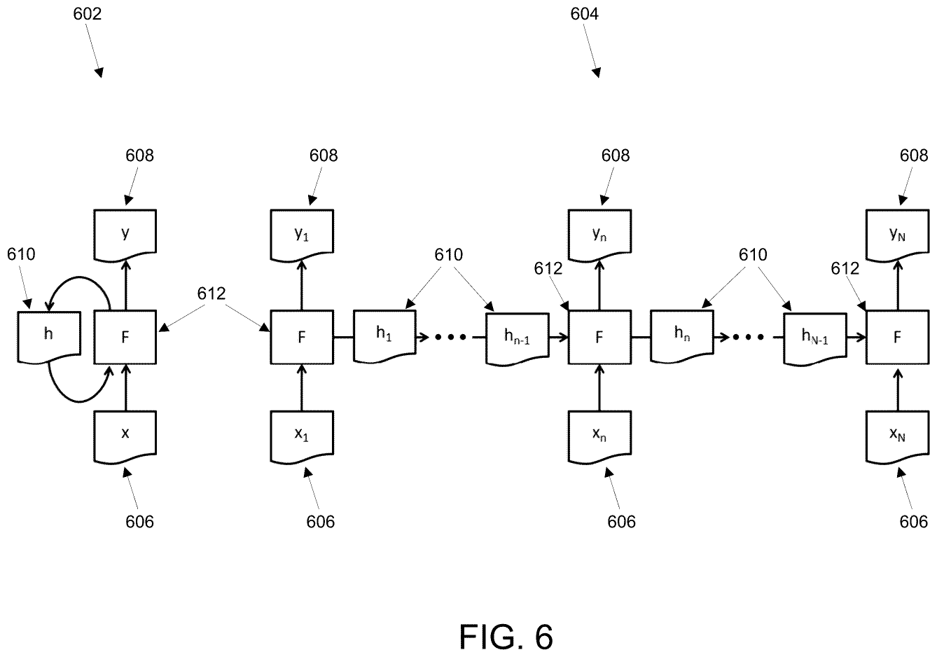

FIG. 6 shows the schematic structure of a recurrent machine learning model F, both in a recurrent representation 602 and in an unfolded representation 604, that may be used to implement one or more machine learning models described herein. The recurrent machine learning model takes as input several input datasets x, x₁, . . . , x_{N} 606 and creates a corresponding set of output datasets y, y₁, . . . , y_{N} 608. Furthermore, the output depends on a so-called hidden vector h, h₁, . . . , h_{N} 610, which implicitly comprises information about input datasets previously used as input for the recurrent machine learning model F 612. By using these hidden vectors h, h₁, . . . , h_{N} 610, a sequentiality of the input datasets can be leveraged.

In a single step of the processing, the recurrent machine learning model F 612 takes as input the hidden vector h_{N-1} created within the previous step and an input dataset xₙ. Within this step, the recurrent machine learning model F generates as output an updated hidden vector h_{N} and an output dataset yₙ. In other words, one step of processing calculates (yₙ, hₙ)=F (xₙ, hₙ₋₁), or by splitting the recurrent machine learning model F 612 into a part F(y) calculating the output data and F(h) calculating the hidden vector, one step of processing calculates yₙ=F^{(y)}(xₙ, hₙ₋₁) and hₙ=F^{(h)}(xₙ, hₙ₋₁). For the first processing step, h₀ can be chosen randomly or filled with all entries being zero. The parameters of the recurrent machine learning model F 612 that were trained based on training datasets before do not change between the different processing steps.

In particular, the output data and the hidden vector of a processing step depend on all the previous input datasets used in the previous steps. yₙ=F^{(y)}(xₙ, F^{(h)}(xₙ₋₁, hₙ₋₂)) and hₙ=F(h)(xₙ, F^{(h)}(xₙ₋₁, hₙ₋₂)).

Systems, apparatuses, and methods described herein may be implemented using digital circuitry, or using one or more computers using well-known computer processors, memory units, storage devices, computer software, and other components. Typically, a computer includes a processor for executing instructions and one or more memories for storing instructions and data. A computer may also include, or be coupled to, one or more mass storage devices, such as one or more magnetic disks, internal hard disks and removable disks, magneto-optical disks, optical disks, etc. Systems, apparatuses, and methods described herein may be implemented using computers operating in a client-server relationship. Typically, in such a system, the client computers are located remotely from the server computer and interact via a network. The client-server relationship may be defined and controlled by computer programs running on the respective client and server computers.

Systems, apparatuses, and methods described herein may be implemented within a network-based cloud computing system. In such a network-based cloud computing system, a server or another processor that is connected to a network communicates with one or more client computers via a network. A client computer may communicate with the server via a network browser application residing and operating on the client computer, for example. A client computer may store data on the server and access the data via the network. A client computer may transmit requests for data, or requests for online services, to the server via the network. The server may perform requested services and provide data to the client computer(s). The server may also transmit data adapted to cause a client computer to perform a specified function, e.g., to perform a calculation, to display specified data on a screen, etc. For example, the server may transmit a request adapted to cause a client computer to perform one or more of the steps or functions of the methods and workflows described herein, including one or more of the steps or functions of FIGS. 1-3. Certain steps or functions of the methods and workflows described herein, including one or more of the steps or functions of FIGS. 1-3, may be performed by a server or by another processor in a network-based cloud-computing system. Certain steps or functions of the methods and workflows described herein, including one or more of the steps of FIGS. 1-3, may be performed by a client computer in a network-based cloud computing system. The steps or functions of the methods and workflows described herein, including one or more of the steps of FIGS. 1-3, may be performed by a server and/or by a client computer in a network-based cloud computing system, in any combination.

Systems, apparatuses, and methods described herein may be implemented using a computer program product tangibly embodied in an information carrier, e.g., in a non-transitory machine-readable storage device, for execution by a programmable processor; and the method and workflow steps described herein, including one or more of the steps or functions of FIGS. 1-3, may be implemented using one or more computer programs that are executable by such a processor. A computer program is a set of computer program instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

A high-level block diagram of an example computer 702 that may be used to implement systems, apparatuses, and methods described herein is depicted in FIG. 7. Computer 702 includes a processor 704 operatively coupled to a data storage device 712 and a memory 710. Processor 704 controls the overall operation of computer 702 by executing computer program instructions that define such operations. The computer program instructions may be stored in data storage device 712, or other computer readable medium, and loaded into memory 710 when execution of the computer program instructions is desired. Thus, the method and workflow steps or functions of FIGS. 1-3 can be defined by the computer program instructions stored in memory 710 and/or data storage device 712 and controlled by processor 704 executing the computer program instructions. For example, the computer program instructions can be implemented as computer executable code programmed by one skilled in the art to perform the method and workflow steps or functions of FIGS. 1-3. Accordingly, by executing the computer program instructions, the processor 704 executes the method and workflow steps or functions of FIGS. 1-3. Computer 702 may also include one or more network interfaces 706 for communicating with other devices via a network. Computer 702 may also include one or more input/output devices 708 that enable user interaction with computer 702 (e.g., display, keyboard, mouse, speakers, buttons, etc.).

Processor 704 may include both general and special purpose microprocessors, and may be the sole processor or one of multiple processors of computer 702. Processor 704 may include one or more central processing units (CPUs), for example. Processor 704, data storage device 712, and/or memory 710 may include, be supplemented by, or incorporated in, one or more application-specific integrated circuits (ASICs) and/or one or more field programmable gate arrays (FPGAs).

Data storage device 712 and memory 710 each include a tangible non-transitory computer readable storage medium. Data storage device 712, and memory 710, may each include high-speed random access memory, such as dynamic random access memory (DRAM), static random access memory (SRAM), double data rate synchronous dynamic random access memory (DDR RAM), or other random access solid state memory devices, and may include non-volatile memory, such as one or more magnetic disk storage devices such as internal hard disks and removable disks, magneto-optical disk storage devices, optical disk storage devices, flash memory devices, semiconductor memory devices, such as erasable programmable read-only memory (EPROM), electrically erasable programmable read-only memory (EEPROM), compact disc read-only memory (CD-ROM), digital versatile disc read-only memory (DVD-ROM) disks, or other non-volatile solid state storage devices.

Input/output devices 708 may include peripherals, such as a printer, scanner, display screen, etc. For example, input/output devices 708 may include a display device such as a cathode ray tube (CRT) or liquid crystal display (LCD) monitor for displaying information to the user, a keyboard, and a pointing device such as a mouse or a trackball by which the user can provide input to computer 702.

An image acquisition device 714 can be connected to the computer 702 to input image data (e.g., medical images) to the computer 702. It is possible to implement the image acquisition device 714 and the computer 702 as one device. It is also possible that the image acquisition device 714 and the computer 702 communicate wirelessly through a network. In a possible embodiment, the computer 702 can be located remotely with respect to the image acquisition device 714.

Any or all of the systems, apparatuses, and methods discussed herein may be implemented using one or more computers such as computer 702.

One skilled in the art will recognize that an implementation of an actual computer or computer system may have other structures and may contain other components as well, and that FIG. 7 is a high level representation of some of the components of such a computer for illustrative purposes.

Independent of the grammatical term usage, individuals with male, female or other gender identities are included within the term.

The foregoing Detailed Description is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the principles of the present invention and that various modifications may be implemented by those skilled in the art without departing from the scope and spirit of the invention. Those skilled in the art could implement various other feature combinations without departing from the scope and spirit of the invention.

The following is a list of non-limiting illustrative embodiments disclosed herein:
Illustrative embodiment 1. A computer-implemented method comprising: receiving medical data of a patient; extracting features from the medical data using at least one of a machine learning based image encoder or a machine learning based text encoder; performing one or more medical imaging analysis tasks based on the extracted features using one or more machine learning based decoders; and outputting results of the one or more medical imaging analysis tasks, wherein the at least one of the machine learning based image encoder or the machine learning based text encoder are trained by: receiving image/text pairs each comprising a training medical image and training text-based medical data; encoding the training medical images into image features using the machine learning based image encoder; encoding the training text-based medical data into text features using the machine learning based text encoder; extracting clinical labels from the training text-based medical data using one or more language models; and training the at least one of the machine learning based image encoder or the machine learning based text encoder based on the image features, the text features, and the clinical labels.
Illustrative embodiment 2. The computer-implemented method of illustrative embodiment 1, wherein training the at least one of the machine learning based image encoder or the machine learning based text encoder based on the image features, the text features, and the clinical labels comprises: computing a clinical context loss by clustering the image features and the text features of each of the image/text pairs based on the clinical labels.
Illustrative embodiment 3. The computer-implemented method of illustrative embodiment 2, wherein training the at least one of the machine learning based image encoder or the machine learning based text encoder based on the image features, the text features, and the clinical labels further comprises: computing a contrastive learning loss by: minimizing a distance between the image features and the text features from the same image/text pair; and maximizing distances between the image features and the text features from different ones of the image/text pairs.
Illustrative embodiment 4. The computer-implemented method of illustrative embodiment 3, wherein training the at least one of the machine learning based image encoder or the machine learning based text encoder based on the image features, the text features, and the clinical labels further comprises: jointly optimizing the clinical context loss and the contrastive learning loss.
Illustrative embodiment 5. The computer-implemented method of any one of illustrative embodiments 1-4, wherein extracting clinical labels from the training text-based medical data using one or more language models comprises: extracting clinically relevant sentences from the training text-based medical data; and determining the clinical labels for the clinically relevant sentences.
Illustrative embodiment 6. The computer-implemented method of any one of illustrative embodiments 1-5, wherein: extracting clinically relevant sentences from the training text-based medical data comprises extracting the clinically relevant sentences from the training text-based medical data using a first language model; and determining the clinical labels for the clinically relevant sentences comprises determining the clinical labels for the clinically relevant sentences using a second language model.
Illustrative embodiment 7. An apparatus comprising: means for receiving medical data of a patient; means for extracting features from the medical data using at least one of a machine learning based image encoder or a machine learning based text encoder; means for performing one or more medical imaging analysis tasks based on the extracted features using one or more machine learning based decoders; and means for outputting results of the one or more medical imaging analysis tasks, wherein the at least one of the machine learning based image encoder or the machine learning based text encoder are trained by: receiving image/text pairs each comprising a training medical image and training text-based medical data; encoding the training medical images into image features using the machine learning based image encoder; encoding the training text-based medical data into text features using the machine learning based text encoder; extracting clinical labels from the training text-based medical data using one or more language models; and training the at least one of the machine learning based image encoder or the machine learning based text encoder based on the image features, the text features, and the clinical labels.
Illustrative embodiment 8. The apparatus of illustrative embodiment 7, wherein training the at least one of the machine learning based image encoder or the machine learning based text encoder based on the image features, the text features, and the clinical labels comprises: computing a clinical context loss by clustering the image features and the text features of each of the image/text pairs based on the clinical labels.
Illustrative embodiment 9. The apparatus of illustrative embodiment 8, wherein training the at least one of the machine learning based image encoder or the machine learning based text encoder based on the image features, the text features, and the clinical labels further comprises: computing a contrastive learning loss by: minimizing a distance between the image features and the text features from the same image/text pair;
and maximizing distances between the image features and the text features from different ones of the image/text pairs.
Illustrative embodiment 10. The apparatus of illustrative embodiment 9, wherein training the at least one of the machine learning based image encoder or the machine learning based text encoder based on the image features, the text features, and the clinical labels further comprises: jointly optimizing the clinical context loss and the contrastive learning loss.
Illustrative embodiment 11. The apparatus of any one of illustrative embodiments 7-10, wherein extracting clinical labels from the training text-based medical data using one or more language models comprises: extracting clinically relevant sentences from the training text-based medical data; and determining the clinical labels for the clinically relevant sentences.
Illustrative embodiment 12. The apparatus of any one of illustrative embodiments 7-11, wherein: extracting clinically relevant sentences from the training text-based medical data comprises extracting the clinically relevant sentences from the training text-based medical data using a first language model; and determining the clinical labels for the clinically relevant sentences comprises determining the clinical labels for the clinically relevant sentences using a second language model.
Illustrative embodiment 13. A non-transitory computer-readable storage medium comprising instructions which, when executed by a computer, cause the computer to carry out operations comprising: receiving medical data of a patient; extracting features from the medical data using at least one of a machine learning based image encoder or a machine learning based text encoder; performing one or more medical imaging analysis tasks based on the extracted features using one or more machine learning based decoders; and outputting results of the one or more medical imaging analysis tasks, wherein the at least one of the machine learning based image encoder or the machine learning based text encoder are trained by: receiving image/text pairs each comprising a training medical image and training text-based medical data; encoding the training medical images into image features using the machine learning based image encoder; encoding the training text-based medical data into text features using the machine learning based text encoder; extracting clinical labels from the training text-based medical data using one or more language models; and training the at least one of the machine learning based image encoder or the machine learning based text encoder based on the image features, the text features, and the clinical labels.
Illustrative embodiment 14. The non-transitory computer-readable storage medium of illustrative embodiment 13, wherein training the at least one of the machine learning based image encoder or the machine learning based text encoder based on the image features, the text features, and the clinical labels comprises: computing a clinical context loss by clustering the image features and the text features of each of the image/text pairs based on the clinical labels.
Illustrative embodiment 15. The non-transitory computer-readable storage medium of illustrative embodiment 14, wherein training the at least one of the machine learning based image encoder or the machine learning based text encoder based on the image features, the text features, and the clinical labels further comprises: computing a contrastive learning loss by: minimizing a distance between the image features and the text features from the same image/text pair; and maximizing distances between the image features and the text features from different ones of the image/text pairs.
Illustrative embodiment 16. The non-transitory computer-readable storage medium of illustrative embodiment 15, wherein training the at least one of the machine learning based image encoder or the machine learning based text encoder based on the image features, the text features, and the clinical labels further comprises: jointly optimizing the clinical context loss and the contrastive learning loss.
Illustrative embodiment 17. The non-transitory computer-readable storage medium of any one of illustrative embodiments 13-16, wherein extracting clinical labels from the training text-based medical data using one or more language models comprises: extracting clinically relevant sentences from the training text-based medical data; and determining the clinical labels for the clinically relevant sentences.
Illustrative embodiment 18. The non-transitory computer-readable storage medium of any one of illustrative embodiments 13-17, wherein: extracting clinically relevant sentences from the training text-based medical data comprises extracting the clinically relevant sentences from the training text-based medical data using a first language model; and determining the clinical labels for the clinically relevant sentences comprises determining the clinical labels for the clinically relevant sentences using a second language model.
Illustrative embodiment 19. A computer-implemented method comprising: receiving image/text pairs each comprising a training medical image and training text-based medical data; encoding the training medical images into image features using a machine learning based image encoder; encoding the training text-based medical data into text features using a machine learning based text encoder; extracting clinical labels from the training text-based medical data using one or more language models; and training at least one of the machine learning based image encoder or the machine learning based text encoder based on the image features, the text features, and the clinical labels.
Illustrative embodiment 20. The computer-implemented method of illustrative embodiment 19, wherein training at least one of the machine learning based image encoder or the machine learning based text encoder based on the image features, the text features, and the clinical labels comprises: computing a clinical context loss by clustering the image features and the text features of each of the image/text pairs based on the clinical labels.

1. A computer-implemented method comprising:
receiving medical data of a patient;
extracting features from the medical data using at least one of a machine learning based image encoder or a machine learning based text encoder;
performing one or more medical imaging analysis tasks based on the extracted features using one or more machine learning based decoders; and
outputting results of the one or more medical imaging analysis tasks,
wherein the at least one of the machine learning based image encoder or the machine learning based text encoder are trained by:
receiving image/text pairs each comprising a training medical image and training text-based medical data;
encoding the training medical images into image features using the machine learning based image encoder;
encoding the training text-based medical data into text features using the machine learning based text encoder;
extracting clinical labels from the training text-based medical data using one or more language models; and
training the at least one of the machine learning based image encoder or the machine learning based text encoder based on the image features, the text features, and the clinical labels.

2. The computer-implemented method of claim 1, wherein training the at least one of the machine learning based image encoder or the machine learning based text encoder based on the image features, the text features, and the clinical labels comprises:
computing a clinical context loss by clustering the image features and the text features of each of the image/text pairs based on the clinical labels.

3. The computer-implemented method of claim 2, wherein training the at least one of the machine learning based image encoder or the machine learning based text encoder based on the image features, the text features, and the clinical labels further comprises:
computing a contrastive learning loss by:
minimizing a distance between the image features and the text features from the same image/text pair; and
maximizing distances between the image features and the text features from different ones of the image/text pairs.

4. The computer-implemented method of claim 3, wherein training the at least one of the machine learning based image encoder or the machine learning based text encoder based on the image features, the text features, and the clinical labels further comprises:
jointly optimizing the clinical context loss and the contrastive learning loss.

5. The computer-implemented method of claim 1, wherein extracting clinical labels from the training text-based medical data using one or more language models comprises:
extracting clinically relevant sentences from the training text-based medical data; and
determining the clinical labels for the clinically relevant sentences.

6. The computer-implemented method of claim 1, wherein:
extracting clinically relevant sentences from the training text-based medical data comprises extracting the clinically relevant sentences from the training text-based medical data using a first language model; and
determining the clinical labels for the clinically relevant sentences comprises determining the clinical labels for the clinically relevant sentences using a second language model.

7. An apparatus comprising:
means for receiving medical data of a patient;
means for extracting features from the medical data using at least one of a machine learning based image encoder or a machine learning based text encoder;
means for performing one or more medical imaging analysis tasks based on the extracted features using one or more machine learning based decoders; and
means for outputting results of the one or more medical imaging analysis tasks,
wherein the at least one of the machine learning based image encoder or the machine learning based text encoder are trained by:
receiving image/text pairs each comprising a training medical image and training text-based medical data;
encoding the training medical images into image features using the machine learning based image encoder;
encoding the training text-based medical data into text features using the machine learning based text encoder;
extracting clinical labels from the training text-based medical data using one or more language models; and
training the at least one of the machine learning based image encoder or the machine learning based text encoder based on the image features, the text features, and the clinical labels.

8. The apparatus of claim 7, wherein training the at least one of the machine learning based image encoder or the machine learning based text encoder based on the image features, the text features, and the clinical labels comprises:
computing a clinical context loss by clustering the image features and the text features of each of the image/text pairs based on the clinical labels.

9. The apparatus of claim 8, wherein training the at least one of the machine learning based image encoder or the machine learning based text encoder based on the image features, the text features, and the clinical labels further comprises:
computing a contrastive learning loss by:
minimizing a distance between the image features and the text features from the same image/text pair; and
maximizing distances between the image features and the text features from different ones of the image/text pairs.

10. The apparatus of claim 9, wherein training the at least one of the machine learning based image encoder or the machine learning based text encoder based on the image features, the text features, and the clinical labels further comprises:
jointly optimizing the clinical context loss and the contrastive learning loss.

11. The apparatus of claim 7, wherein extracting clinical labels from the training text-based medical data using one or more language models comprises:
extracting clinically relevant sentences from the training text-based medical data; and
determining the clinical labels for the clinically relevant sentences.

12. The apparatus of claim 7, wherein:
extracting clinically relevant sentences from the training text-based medical data comprises extracting the clinically relevant sentences from the training text-based medical data using a first language model; and
determining the clinical labels for the clinically relevant sentences comprises determining the clinical labels for the clinically relevant sentences using a second language model.

13. A non-transitory computer-readable storage medium comprising instructions which, when executed by a computer, cause the computer to carry out operations comprising:
receiving medical data of a patient;
extracting features from the medical data using at least one of a machine learning based image encoder or a machine learning based text encoder;
performing one or more medical imaging analysis tasks based on the extracted features using one or more machine learning based decoders; and
outputting results of the one or more medical imaging analysis tasks,
wherein the at least one of the machine learning based image encoder or the machine learning based text encoder are trained by:
receiving image/text pairs each comprising a training medical image and training text-based medical data;
encoding the training medical images into image features using the machine learning based image encoder;
encoding the training text-based medical data into text features using the machine learning based text encoder;
extracting clinical labels from the training text-based medical data using one or more language models; and
training the at least one of the machine learning based image encoder or the machine learning based text encoder based on the image features, the text features, and the clinical labels.

14. The non-transitory computer-readable storage medium of claim 13, wherein training the at least one of the machine learning based image encoder or the machine learning based text encoder based on the image features, the text features, and the clinical labels comprises:
computing a clinical context loss by clustering the image features and the text features of each of the image/text pairs based on the clinical labels.

15. The non-transitory computer-readable storage medium of claim 14, wherein training the at least one of the machine learning based image encoder or the machine learning based text encoder based on the image features, the text features, and the clinical labels further comprises:
computing a contrastive learning loss by:
minimizing a distance between the image features and the text features from the same image/text pair; and
maximizing distances between the image features and the text features from different ones of the image/text pairs.

16. The non-transitory computer-readable storage medium of claim 15, wherein training the at least one of the machine learning based image encoder or the machine learning based text encoder based on the image features, the text features, and the clinical labels further comprises:
jointly optimizing the clinical context loss and the contrastive learning loss.

17. The non-transitory computer-readable storage medium of claim 13, wherein extracting clinical labels from the training text-based medical data using one or more language models comprises:
extracting clinically relevant sentences from the training text-based medical data; and
determining the clinical labels for the clinically relevant sentences.

18. The non-transitory computer-readable storage medium of claim 13, wherein:
extracting clinically relevant sentences from the training text-based medical data comprises extracting the clinically relevant sentences from the training text-based medical data using a first language model; and
determining the clinical labels for the clinically relevant sentences comprises determining the clinical labels for the clinically relevant sentences using a second language model.

19. A computer-implemented method comprising:
receiving image/text pairs each comprising a training medical image and training text-based medical data;
encoding the training medical images into image features using a machine learning based image encoder;
encoding the training text-based medical data into text features using a machine learning based text encoder;
extracting clinical labels from the training text-based medical data using one or more language models; and
training at least one of the machine learning based image encoder or the machine learning based text encoder based on the image features, the text features, and the clinical labels.

20. The computer-implemented method of claim 19, wherein training at least one of the machine learning based image encoder or the machine learning based text encoder based on the image features, the text features, and the clinical labels comprises:
computing a clinical context loss by clustering the image features and the text features of each of the image/text pairs based on the clinical labels.
